(19) United States
(12) Patent Application Publication
(10) Pub. No.: US 2022/0000393 A1
(43) Pub. Date: Jan. 6, 2022

(54) SYSTEMS AND METHODS FOR IMPROVING PHYSICAL PERFORMANCE

(71) Applicant: Savitar, Inc., Flushing, NY (US)

(72) Inventor: Qasim ASHRAF, Flushing, NY (US)

(73) Assignee: Savitar, Inc., Flushing, NY (US)

(21) Appl. No.: 17/368,006

(22) Filed: Jul. 6, 2021

Related U.S. Application Data
(60) Provisional application No. 63/048,341, filed on Jul. 6, 2020.

(51) Int. Cl. A61B 5/11; A61B 5/00

(52) U.S. Cl. CPC A61B 5/112; A61B 5/1116; A61B 5/7264; A61B 5/0004; A61B 2562/0247; A61B 5/7405; A61B 2503/10; A61B 2505/09; A61B 2562/0219; A61B 5/6807

(57) ABSTRACT

Systems and methods are described herein for improving walking or running performance in a variety of settings, including athletic training and physical therapy settings. Software programs comprising machine learning algorithms are employed with the objective of synthesizing real-time, actionable recommendations for performance improvement based on data inputs from a plurality of sensors incorporated into footwear. The recommendations may be visualized or heard during and after performance, either on a mobile computing device such as a smartphone or smartwatch or over a web interface on a home computer, and historical performance data may be incorporated into a personalized database for each user. The systems include real-time recommendations for changes in form and personalized coaching such as of athletes seeking to improve performance. The systems and methods described herein may also be employed for the purposes of injury prevention or rehabilitation in a medical setting.

### CROSS-REFERENCE TO RELATED APPLICATIONS

This application claims the benefit of priority of U.S. Provisional Patent Application 63/048,341, filed Jul. 6, 2020, the contents of which are incorporated herein by reference in their entirety.

### STATEMENT REGARDING FEDERALLY SPONSORED RESEARCH OR DEVELOPMENT

The invention described herein was conceived and reduced to practice without the benefit of federal funding.

### BACKGROUND OF THE INVENTION

Modern athletic and functional footwear are available in a wide array of designs suitable for many different sports, activities and other uses. With the advent of sensing technologies, Bluetooth, software applications and the like, some shoe companies have developed shoes that incorporate some such technical components to track steps, distances traveled, and other metrics associated with walking, jogging, running, playing sports, or rehabilitative activities, which may then be readable on a smartphone, tablet or the like in communication with the sensors installed in or on the sneakers or elsewhere.

While these products represent innovations from traditional footwear lacking in any sensing or electronic features, these products have not been designed to track form, gait, pressure distribution, center of balance, speed and many other metrics that could be very useful to the people wearing them. Indeed, none of the current solutions integrate pressure sensors into a system for tracking these important parameters, for example. Thus, medically useful data related to stride and gait, for example, are not measured by current methods. There are currently no footwear on the market that track all these data points and give actionable insight and feedback on how to improve, which is one object of the invention of the present disclosure.

Currently, the products on the market that come closest to achieving the objectives described in connection with the present invention are quantitative fitness trackers that execute step counts, but without providing any information on how the steps were taken and how to improve. More recent products include a line of sneakers that has some functionality in the sneaker (cadence tracking with an acceleration/gyroscope module), but these products lack accuracy and detailed output. Similar products operate through an insole and clip-on feature, which provides less reliable data and a poor user experience. Therefore, there is a need in the art for systems and methods that can leverage signals from pressure sensors and other inputs in order to help athletes and others improve their form through the use of algorithms and machine learning.

### SUMMARY OF THE INVENTION

Described herein are systems and methods that harness inputs from pressure sensors integrated into sneakers or parts thereof, synthesize information from these inputs, and provide excellent user experience and feedback on multiple aspects of walking, running and exercising on the part of the wearers. The inclusion of pressure sensors allows the user to obtain medical quality data that has historically only been accessible in a professional setting, such as a fitness laboratory or high quality training facility, while the use of modern computing technologies and software programming enables machine learning outputs that provide users with actionable results to improve their form and ultimately their overall health through real-time coaching; personalized training plans; custom goals; future recommendations for footwear; and workout quality information.

In addition to pressure sensors, a custom sensor sheet of, for example, polyethylene terephthalate (PET), that measures pressures exerted at points throughout the entire foot of the user in correspondence with various anatomical regions of the bottom of the feet of the person wearing a shoe with the sensor sheet incorporated therein. A sensor sheet may comprise an encased unit further comprising one or more of an accelerometer, gyroscope, wireless charging capability, Bluetooth transceiver, flash memory device or equivalent, or combinations thereof, all in network communication with a system that enables not only electronic data collection and storage, but the application of algorithms in order to synthesize recommendations for improvements users can make to their form.

In Strobel lasted embodiments, all of the electrical leads for data packet transmission will optimally reside between the Strobel board and the midsole of the sneaker. In certain embodiments, the midsole will be configured with a cavity that allows the sensor sheet and other features to be installed in the cavity, then the Strobel board and upper will reside above. In preferred embodiments, the outsole of the footwear as described herein may be configured with cavities for housing pressure sensors and other electronic and communications components according to the present disclosure. Other embodiments are possible, as will be evident to one of ordinary skill in the art.

### BRIEF DESCRIPTION OF THE DRAWINGS

FIG. 1 illustrates a system according to an embodiment of the present invention.

FIG. 2 illustrates a schematic of a sensor sheet of footwear according to an embodiment of the present invention.

FIG. 3 is a flow chart illustrating the method steps executed by a processor in accordance with software instructions according to an embodiment of the present invention.

FIG. 4 illustrates an example of a visual display according to the present invention.

### DETAILED DESCRIPTION OF ILLUSTRATIVE EMBODIMENTS

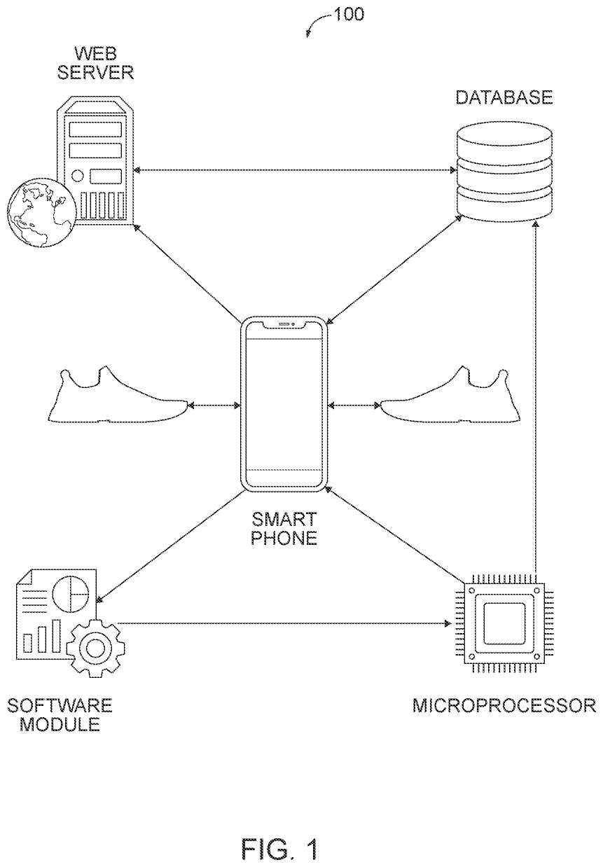

Illustrative embodiments of the invention of the present disclosure are best described with reference to the accompanying drawings. FIG. 1 illustrates an embodiment of a system according to the present disclosure 100, in which a smartphone, for example, receives inputs from pressure sensors residing within footwear and configured to acquire and transmit various types of data informative of the walking, jogging or running technique of a person wearing the footwear. System inputs originate from a plurality of sensors, such as pressure sensors, as will be familiar to one of ordinary skill in the art and are converted from electrical (analog) to logical (digital) signals for processing.

The transmission of logical signals, e.g., in the form of data packets, may be using various wireless communications protocols such as Bluetooth, as will be familiar to one of ordinary skill in the art. Data may be received by a compatible device such as a smartphone or smartwatch equipped with a suitable application programming interface (API) for processing by a microprocessor in accordance with instructions provided in a software program tangibly stored on a non-transitory computer readable medium, such as a cloud server. The raw and processed data may be stored locally or remotely within a database for additional processing.

As illustrated in FIG. 1, a smart phone, by way of example and not limitation, carried by a user of the system during a monitoring period such as a walk, run, jog or physical therapy session collects information from footwear as described herein, and communicates that information to a software module tangibly stored on a non-transitory computer readable medium comprising instructions which when executed by a microprocessor cause the microprocessor to store the information, tagged according to input, processed and unprocessed, in a connected database, an also feed the processed information back to the collecting device such that a user may obtain real-time feedback on physical performance. Additionally, the mobile (smartphone) device will be in network communication with a web server, as will the database, such that synchronized communications among the system elements may be achieved to enable the methods that are objects of the present invention, namely multiple methods of improving performance when running, jogging, walking or participating in sporting events or physical therapy sessions.

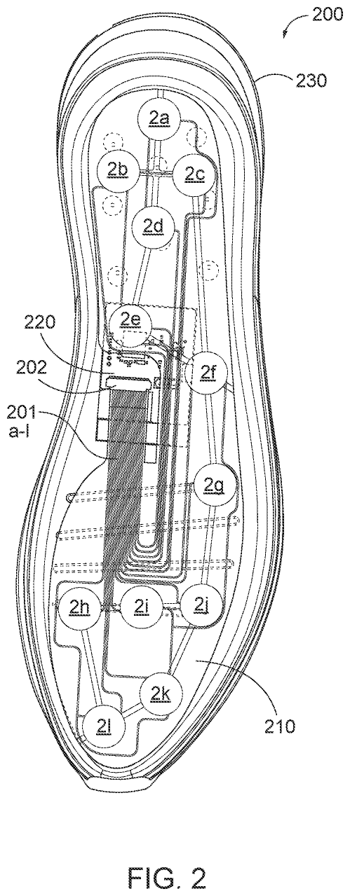

Exemplary sensor positions and circuit schematics are illustrated in FIG. 2, which shows an embodiment of a sensor sheet 210 positioned above a midsole or outsole 230 of footwear as described herein 200. In preferred embodiments, each individual sensor may be configured with a stiffener to better ensure accurate readings in response to the strides of a wearer of a sneaker of the present invention. As can be seen in FIG. 2, a shoe according to the present invention is ideally configured with multiple pressure sensors at strategic locations within a sensor sheets each corresponding to pressure points expected to be activated during walking or running in a shoe comprising the sensor sheet. The illustrative embodiment shown is configured with 12 pressure sensors and circuitry enabling centralized collection of outputs. In this illustrative embodiment, 12 pressure sensors with logical connections are enabled to gather raw data from the various sensors. Data is then stored on memory, which is transmitted via Bluetooth to a computing device such as a smartphone or smartwatch, which in turn transmits via wireless network such as Wi-Fi to a cloud server. From there, raw data is processed in accordance with provided software program instructions tangibly stored on a non-transitory computer readable medium as will be familiar to one of ordinary skill in the art. The thickness of a sensor sheet may be four mils according to an exemplary embodiment, with a depression on the outsole into which a sensor sheet may fit for bonding.

In the illustrative embodiment of FIG. 2, four sensors (2a-2d) are positioned about the heel area of the footwear, sensor 2e is positioned inside the arc of the foot, sensor 2f is positioned at the outside of the arc of the foot, sensor 2g is positioned between sensor 2f and the posterior region of the tarsals (2h-2j), including sensor 2h positioned at the ball of the foot, with sensor 2l positioned at the big toe and sensor 2k centrally located among the remaining toes. Electrical leads 201a-l originate at sensors 2a-2l, respectively, and converge at a central collection point 202 positioned ideally within a cavity 220 of a shoe midsole or outsole comprising local memory and transceiver, where electrical signals from the sensors may be converted to data packets, stored, and transmitted to a local mobile computing device.

In preferred embodiments, all data and processing outputs are visible on a user interface of a mobile software application installed on a smartphone, smartwatch or the like. Through a user interface, such as a graphical user interface (GUI), users may view the analytical output of how they ran or walked during a selected time period (e.g., medical gait analysis, speed, cadence, stride length, foot fall), and may then endeavor to make corrective adjustments to gait and posture, for example, based on the analytical output. As illustrated in FIG. 2, pressure sensors may be selectively placed to correspond to various anatomical features of the foot of the wearer of a pressure sensor sneaker as described herein. One familiar with the field of podiatry will understand that forward sensors may be placed proximally to the various tarsals of the foot in order to better understand those parts of the foot anatomy that are being correctly or incorrectly subjected to certain levels of pressure.

For example, sensors may be placed proximal to the metatarsals or ball of the foot to detect how much pressure is being applied there and adjust gait to apply more pressure rearward if too much pressure is being exerted on the ball of the foot. Similarly, forward sensors may be placed proximal to the phalanges to determine whether the correct amount of pressure is being applied as would be expected there when walking or running. Sensors may be located within the arc of the foot such as below the cuneiform or navicular tarsals to detect flat-footed running, for example. Sensors may likewise be located beneath the heel and ankle in order to detect undue pressure being applied to that area during a run.

The convergence of electrical connections provides an example of convergence of logical signals prior to Bluetooth transmission of raw data via Wi-Fi or other suitable wireless network technology to a cloud server in communication with a smartphone or the like equipped with a mobile software application for data processing and summary of performance of a given run, jog, walk or trek, for example. One can then view on a provided GUI performance data to include the distribution of pressure across the feet during a selected time period. Alternatively, a web application may be provided so a user can access the data from a home computer in network connection with the system outputs for more comfortable assessment and analysis. As depicted in FIG. 2, sensors 2a-2d may track heel lift for example, while sensor 2e tracks arch, with sensors 2h-2j tracking lift angle.

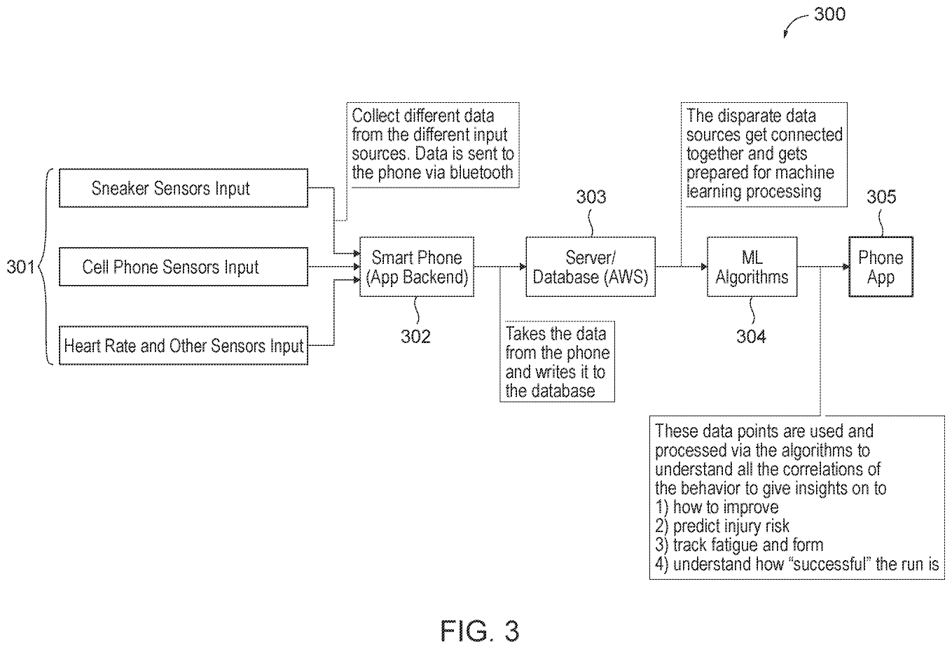

Turning now to FIG. 3, a flow chart 300 is provided showing the various signal inputs 301 being transmitted via Bluetooth or the like to a nearby smartphone 302 application, for example and not by way of limitation. Data packets that are then transmitted via wireless network to a web server 303 for storage and processing may then be tagged according to the originating sensor and time of collection such that changes in pressures exerted during use over time may be monitored. Disparate sensor data may be aggregated and processed for visualization in accordance with provided software algorithms, but may also be combined and processed further, collectively, to produce synthetic outputs representative of machine learning 304 and fed back into the smartphone app 305. In this manner, a user is provided with real-time feedback on how to improve form, avoid injury, avoid fatigue and understand how successful the outing was, be it a run, jog, walk or physical therapy session.

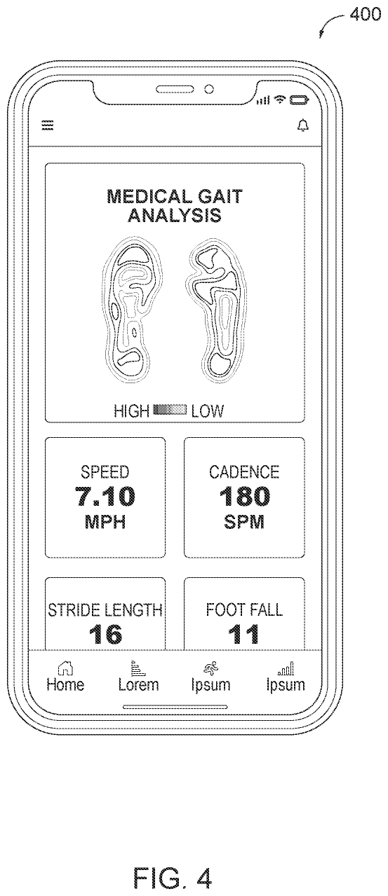

Turning to FIG. 4, an illustration of an exemplary GUI 400 according to the present invention is provided, showing processed data inputs from various sensor collection points at various times may be used to assess the form of a user and changes in form over the duration of a selected data collection period. For example, the machine learning algorithms according to the present invention may be utilized to adjust form and prevent injury by tracking changes in speed, cadence, stride length and foot fall that result in undesirable amounts of pressure concentrated on various parts of the foot anatomy that can lead to injury to the foot or other parts of the leg. In preferred embodiments, a system as described herein may provide real-time coaching to a user, alerting the user to undesirable form or changes in form that are likely to lead to fatigue or injury before they do.

Exemplary dimensions of a shoe sole suitable for housing a sensor sheet as shown in FIG. 2 210, provided for illustrative purposes only, include a heel counter running a vertical distance of 78 mm from the rideliner to the top of the heel collar, forming a heel tab above and to the rear of the shoe. This distance is reduced going from rear to front down to 45 millimeters to form a semicircular space to accommodate the ankle bones of the wearer. The exemplary embodiment 230 shown in FIG. 2 may be configured, for example, with a heel collar of 15 millimeters. From the bottom of the outsole to the top of the rideliner, a distance of 38 millimeters is selected for example, with the rear of the rideliner accounting for half the distance. The rear bottom of the outsole may rise front to rear to be approximately 7 millimeters from the ground surface, while at the front of the outsole that vertical distance is 20 millimeters. Similarly, the distance from the top of the rideliner to the bottom of the outsole may be reduced at the point where the outsole begins to rise toward the front of the shoe from the ground. Other sizes and configurations are possible as will be appreciated by one of ordinary skill in the art.

Any exemplary outsole typically has a widest portion of the sole reaching 85 millimeters across the shoe, reducing down to 60 millimeters at the insole, then expanding out to reach 110 millimeters at the widest portion of the front of the shoe. The overall length of any embodiment will be designed to provide a custom fit for the wearer and achieve the desired pressure points for sensing.

A system as described herein may enable methods designed to improve running performance and the like while preventing injuries as well. Improved gait and posture may be leveraged to prevent injuries such as ankle sprains, plantar fasciitis, runner's knee, Achilles tendinitis, shin splints, stress fractures and other problems associated with running without proper form. Common problems that can be avoided using the invention of the present disclosure include improper cadence, heel striking, high or low stride frequency, improper hip separation, or stiffness. The invention of the present disclosure may also be leveraged to select the proper shoe for running, walking or jogging based on pressure distribution as detected and analyzed according to the methods described herein.

Various layers of a shoe sole including PET sensor sheet are contemplated herein. In exemplary embodiments, a sensor sheet may be bonded between the outsole and midsole or Strobel board of a sneaker as described herein, or configured in another suitable manner. As presented above, an outsole of the present invention may be configured with an indentation of suitable size, shape and depth to accommodate a sensor sheet as described herein and enable the necessary electrical and logical connections for Bluetooth transmission of data to a cloud server of a system as described here, where the raw data obtained by the pressure sensor sheet is processed according to software program instructions tangibly stored on a non-transitory computer readable medium. When executed by a processor, such instructions may be utilized to provide a user with many useful analytical outputs about running or walking form. These processed data may be used to improve running form and avoid injury according to the methods enabled by a pressure sensor sneaker and accompanying system as described herein.

A common system architecture familiar to one of ordinary skill in the art, as shown by way of example in FIG. 1, may be used to enable the storage, maintenance and processing of raw data transmitted via Bluetooth from a sneaker as described herein to the cloud, where a database may be maintained for various types of analyses of interest to a user over time. Data may be accessed, manipulated and processed according to the software instructions developed according to the invention in response to user inputs through an application program interface installed on a smartphone or other wireless communication device configured for communication with the system.

These and other advantages of the present invention will be familiar to those familiar with running, for example, and injury prevention while running. The embodiments described herein are prevented for illustrative purposes and should not be deemed limiting of the capabilities of the systems and methods described herein. In particular, external inputs from other data sources such as heart monitors and other such health monitoring devices may be aggregated with the data collected according to the present disclosure and used to improve the machine learning and artificial intelligence aspects of the disclosure that are all objects of the invention. The main object of the invention is to enable a user of the invention to firstly avoid injury and complications from running, walking or jogging; secondly, to obtain useful advice for improving form and performance; and ultimately, to improve the overall health and well being of the users.

What is claimed is:

1. A system for improving ambulatory and athletic performance, the system comprising:
footwear having a plurality of pressure sensors electrically connected to a transceiver in network communication with a mobile computing device having an application programming interface (API) in communication with a software application tangibly stored on a non-transitory computer readable medium, the software application comprising instructions which when executed by a processor cause the processor to:
record data transmitted from the plurality of sensors via the transceiver in a database, wherein the recorded data includes the identity of each originating sensor; and
synthesize a visual output visible on a graphical user interface (GUI) of the mobile computing device, wherein the visual output represents a quality of an athletic performance of a user of the system.

2. The system of claim 1, wherein the plurality of sensors are integrated into a sensor sheet.

3. The system of claim 1, wherein electrical connections between the plurality of sensors and the transceiver comprise a means for analog to digital signal conversion.

4. The system of claim 3, further comprising a means for local data storage within the footwear.

5. The system of claim 3, further comprising a web application, wherein the web application comprises a GUI on which the visual output is visible.

6. The system of claim 3, wherein the plurality of sensors is selected from the group consisting of a pressure sensor, an altimeter, an accelerometer, a gyrometer, a speedometer, a cadence meter, a stride length meter, a foot fall meter and combinations thereof.

7. The system of claim 6, wherein the positions of the plurality of sensors are collocated with anatomical features of a foot selected from the group consisting of metatarsals, ball of the foot, phalanges, arc of the foot, cuneiform, navicular tarsals, heel, ankle and combinations thereof.

8. The system of claim 7, wherein the mobile computing device generates a real-time audio output audible to a user of the system in response to incoming data from the plurality of sensors, the real-time audio output comprising recommendations to improve the quality of an ambulatory performance of the user.

9. The system of claim 7, wherein the mobile computing device generates a real-time audio output audible to a user of the system in response to incoming data from the plurality of sensors, the real-time audio output comprising recommendations to improve the quality of an athletic performance of the user.

10. The system of claim 7, wherein the mobile computing device generates a real-time audio output audible to a user of the system in response to incoming data from the plurality of sensors, the real-time audio output comprising recommendations to prevent an injury selected from the group consisting of ankle sprains, plantar fasciitis, runner's knee, Achilles tendinitis, shin splints, stress fractures, other problems associated with running without proper form and combinations thereof.

11. A method for improving ambulatory and athletic performance, the method comprising:
wearing the footwear of claim 1 while walking or running;
viewing the visual output visible on the GUI of the mobile computing device; and
altering walking or running form based on observations on the GUI.

12. A method for improving ambulatory and athletic performance, the method comprising:
wearing the footwear of claim 2 while walking or running;
viewing the visual output visible on the GUI of the mobile computing device; and
altering walking or running form based on observations on the GUI.

13. A method for improving ambulatory and athletic performance, the method comprising:
wearing the footwear of claim 3 while walking or running;
viewing the visual output visible on the GUI of the mobile computing device; and
altering walking or running form based on observations on the GUI.

14. A method for improving ambulatory and athletic performance, the method comprising:
wearing the footwear of claim 4 while walking or running;
viewing the visual output visible on the GUI of the mobile computing device; and
altering walking or running form based on observations on the GUI.

15. A method for improving ambulatory and athletic performance, the method comprising:
wearing the footwear of claim 5 while walking or running;
viewing the visual output visible on the GUI of the mobile computing device; and
altering walking or running form based on observations on the GUI.

16. A method for improving ambulatory and athletic performance, the method comprising:
wearing the footwear of claim 6 while walking or running;
viewing the visual output visible on the GUI of the mobile computing device; and
altering walking or running form based on observations on the GUI.

17. A method for improving ambulatory and athletic performance, the method comprising:
wearing the footwear of claim 7 while walking or running;
viewing the visual output visible on the GUI of the mobile computing device; and
altering walking or running form based on observations on the GUI.

18. A method for improving ambulatory and athletic performance, the method comprising:
wearing the footwear of claim 8 while walking or running;
viewing the visual output visible on the GUI of the mobile computing device; and
altering walking or running form based on observations on the GUI.

19. A method for improving ambulatory and athletic performance, the method comprising:
wearing the footwear of claim 9 while walking or running;
viewing the visual output visible on the GUI of the mobile computing device; and
altering walking or running form based on observations on the GUI.

20. A method for improving ambulatory and athletic performance, the method comprising:
wearing the footwear of claim 10 while walking or running;
viewing the visual output visible on the GUI of the mobile computing device; and
altering walking or running form based on observations on the GUI.
